(19) United States
(12) Patent Application Publication
(10) Pub. No.: US 2002/0127204 A1
(43) Pub. Date: Sep. 12, 2002

(54) IMPROVEMENTS IN OR RELATING TO PROTECTION AGAINST INTRACELLULAR INFECTION

(76) Inventor: STEPHEN JAMES RUSSELL, CAMBRIDGE (GB)

Correspondence Address: PALMER & DODGE, LLP, KATHLEEN M. WILLIAMS, 111 HUNTINGTON AVENUE, BOSTON, MA, 02199, US

(21) Appl. No.: 09/051,393

(22) PCT Filed: Oct. 9, 1996
(86) PCT No.: PCT/GB96/02465

(30) Foreign Application Priority Data
Oct. 10, 1995 (GB) 9520641.3

(51) Int. Cl. A61K 48/00; C12N 15/87; C12N 15/00

(52) U.S. Cl. 424/93.1; 435/320.1; 435/325; 435/455; 514/44

(57) ABSTRACT

Disclosed is a method of inhibiting intracellular infection of a eukaryotic cell by a microorganism, comprising introducing into the cell a nucleic acid sequence directing the expression on the surface of the cell of a protective polypeptide receptor molecule which has high binding affinity for a component of a microorganism capable of exsisting intracellularly, such that binding of the microorganism to the protective polypeptide prevents productive infection of the cell by the micoorganism.

### FIELD OF THE INVENTION

The present invention relates to a method of protecting eukaryotic cells against intracellular infection by viruses or other micro-organisms, and to compositions of use in the method.

### BACKGROUND OF THE INVENTION

Retroviral envelope glycoproteins mediate specific viral attachment to cell surface receptors and subsequently trigger fusion between the viral envelope and the target cell membrane. All retroviral envelope spike glycoproteins examined to date are homo-oligomers containing two to four heterodimeric subunits (Doms et al., 1993 Virology 193, p545). Each subunit comprises a large extraviral glycoprotein moiety (SU) noncovalently attached at its C-terminus to a smaller transmembrane polypeptide (TM) that anchors the complex in the viral membrane. The SU and TM polypeptides are derived from a single chain precursor glycoprotein that undergoes proteolytic maturation in the Gogi compartment during its transport to the cell surface. Uncleaved envelope precursor glycoproteins can be incorporated into viruses but are unable to trigger membrane fusion.

Retrovirus attachment triggers virus entry. Different retroviruses have evolved different strategies for transferring their nucleic acid genomes across the limiting membranes of their target cells. A poorly defined cascade of events is triggered when the viral SU glycoprotein attaches to its cognate receptor, culminating in fusion between the lipid membranes of the virus and the host cell. There may be a large number of steps involved in the entry cascade, involving multiple conformational rearrangements in viral and cellular glycoproteins, dissociation of SU glycoprotein subunits, primary and secondary binding reactions, and specific cleavages mediated by cellular proteases (Weiss 1993 In J. Levy (ed.) The Retroviridae. Plenum Press, New York p1-108: Sattentau & Moore 1993 Philos. Trans. R. Soc. Lond. B. Biol. Sci. 343, p59; Weiss & Tailor 1995 Cell 82, p531). Retrovirus attachment is the first step in virus entry, leading to a conformational change in the viral attachment glycoprotein (SU) or its receptor that acts as a highly specific trigger for subsequent events in the entry cascade.

When retrovirus attachment is redirected to a receptor molecule on the cell surface, other than its natural cognate receptor, virus entry may be inefficient. Thus, when ecotropic murine leukaemia virus (MLV)-based retroviral vectors were bound to MHC class I or MHC class II molecules, EGF receptors, insulin receptors or erythropoietin receptors on human cells, gene transfer was inefficient (Etienne-Julan et al., 1992 J. Gen. Virol. 73, p3251; Kasahara et al., 1994 Science 266, p1373). Under certain conditions, HIV entry into human monocytes and macrophages was assisted by bispecific antibodies that could opsonise the virus particles and mediate their attachment to Fc receptors and other cell surface molecules (Connor et al., 1991 PNAS USA 88, p9593). However, infection was completely blocked by anti-CD4 antibodies, indicating that the interaction between gp120 SU and CD4 was required for infectivity, even under conditions of antibody-mediated binding. In another study, complement-mediated HIV binding to CR2 complement receptors on transfected K562 cells (in the absence of CD4) did not allow virus entry (Montefiori et al., 1993 J. Virol. 67, p2699). These results point to the lock and key nature of the initial interaction between a retroviral SU glycoprotein and its receptor as a trigger for virus entry.

Retroviral infection of susceptible target cells can be inhibited using bifunctional crosslinkers that compete the virions away from their natural receptors and bind them to cell surface molecules that do not support efficient virus entry. When bispecific antibodies were used for targeting HIV to monocyte Fc gamma receptors, enhancement of HIV infectivity was only apparent at lower levels of opsonisation; infectivity was reduced when the virus was more heavily opsonised (Connor et al., 1991, cited above). The authors suggested that highly opsonised HIV can initiate high-affinity multivalent interactions with Fc gamma receptors that trigger endocytosis and intracellular degradation of the antibody-virus complex, whereas at lower levels of antibody opsonisation, there are too few interactions with Fc receptors to initiate endocytosis, but there are enough to stabilise the virus at the cell surface, allowing antibody-dependent enhancement of HIV infection through high affinity CD4 interactions.

Retroviral infection of susceptible target cells can also be inhibited by viral incorporation of ligands that compete the virions away from their natural receptors and bind them to cell surface molecules that do not support efficient virus entry. When epidermal growth factor (EGF) was displayed on an amphotropic retroviral vector as an N-terminal extension of the SU glycoprotein, the engineered vector bound preferentially to EGF receptors (rather than to the amphotropic virus receptor—RAM-1) present on EGF receptor-positive human cells and gene transfer did not occur (Cosset et al., 1995 J. Virol. 69, 6314-6322; also WO 96/00294). EGF receptor-negative, RAM-1-positive cells were fully susceptible to the engineered retroviral vector but showed reduced susceptibility when they were genetically modified to express EGF receptors. The reduction in susceptibility was in proportion to the level of EGF receptor expression. Moreover, when soluble EGF was added to competitively inhibit virus capture by the EGF receptors, gene transfer was restored. The loss of infectivity of the EGF-displaying virus on EGF receptor expressing cells is believed to reflect a block to membrane fusion between the viral envelope and the target cell plasma membrane.

Retroviral receptors do not merely provide a binding site for the retroviral SU glycoprotein but provide additional functions that are required for efficient retrovirus entry. Thus, monoclonal antibodies against the V3/V4 domain of the CD4 molecule did not inhibit gp120 binding to CD4 (CD4 binds to the V1 domain), but they did inhibit HIV infection of human PBL (Hasunuma et al., 1992 J. Immunol. 148, p1841). Also, non-HIV-permissive human cells expressing a chimaeric receptor consisting of the first 177 residues of human CD4 attached to residues from the hinge, transmembrane and cytoplasmic domains of human CD8 were defective in their ability to form syncytia with HIV-1-infected cells (Poulin et al., 1991 J. Virol. 65, p4893; Golding et al., 1993 J. Virol. 67, p6469). However, the chimaeric receptor could still trigger gp120 to mediate membrane fusion, although the lag time of membrane fusion was fivefold longer than that for the wild type CD4 molecule. Moreover, these investigators did not co-express the CD8-CD4 chimaeric receptor with wild type CD4 and to date there is no evidence, and has been no suggestion, that a chimaeric receptor might be used to protect an otherwise permissive cell from infection by a virus, or any other micro-organism.

Intracellular diversion of HIV gp160 has been reported as a result of the expression of fusion genes comprising soluble CD4 and lysosome targeting domains, and has been proposed as a potential gene therapy strategy against HIV (Lin et al., 1993 FASEB J. 7, 1070). However, the strategy employs soluble CD4 that is targeted directly to lysosomes and cannot therefore be displayed on the cell surface; it is therefore suitable only for limiting the production of infectious virions by HIV-infected cells, and could not be used to protect a target cell against HIV infection.

### SUMMARY OF THE INVENTION

In a first aspect the invention provides a method of inhibiting intracellular infection of a eukaryotic cell by a micro-organism, comprising introducing into the cell a nucleic acid sequence directing the expression on the surface of the cell of a protective polypeptide receptor molecule which has high binding affinity for a component of a micro-organism capable of existing intracellularly, such that binding of the micro-organism to the protective polypeptide prevents productive infection of the cell by the micro-organism.

The micro-organism is typically a pathogen i.e. a micro-organism capable of causing disease in one or more of plants, animals and humans. Accordingly, the term “micro-organism” as used herein refers to viruses, particularly retroviruses, but also to bacteria (such as Salmonella spp.), protozoa (e.g. Leishmania spp.) and to Rickettsia and Chlamydia. For viruses, the ability to replicate intracellularly is, of course, essential. For many bacteria and protozoa also, the ability to enter certain cell types, to survive and/or multiply within them, is an essential feature of the process by which the micro-organisms cause disease (e.g. survival of Salmonella or Yersinia spp. inside macrophages). It will be appreciated by those skilled in the art that preventing “productive infection of the cell” may be achieved by preventing entry of the micro-organism into the cell and/or by preventing multiplication of the micro-organism within the cell (e.g. by preventing the infecting micro-organism from escaping from degradation within an endosome). Preventing entry of the micro-organism into the cell may conveniently be accomplished, for example, by preventing fusion of an enveloped virus with the host cell membrane.

It will be further understood by those skilled in the art that the protective polypeptide may comprise non-peptide moieties (e.g. sugar residues, lipid and the like) which are commonly associated with proteins).

The method of the invention is particularly useful when applied to, and is desirably performed on, cells which would otherwise be permissive for infection by the micro-organism, such that the cells can be protected against infection.

The concept can be applied to protection against infection by micro-organisms in general, but especially to viruses, whose receptors have the following properties:

a) The micro-organism attachment site is on a membrane protein or glycoprotein;

b) The domain or domains of the receptor protein which carry the micro-organism attachment site can be transplanted onto a heterologous membrane glycoprotein such that the micro-organism attachment function is not disrupted; and

c) The gene coding for the protein that contains the micro-organism attachment site has been cloned.

HIV and measles are examples of viruses that are known to meet all three criteria. The receptors of several other micro-organisms are also well-characterised. However, there are many micro-organisms of clinical/veterinary significance whose receptors have not yet been cloned or characterised in sufficient detail to know whether they will meet the first two criteria. Once fully characterised, those skilled in the art will be well able to develop protective receptor polypeptides for use against these other micro-organisms, with the benefit of the present disclosure.

Of particular interest are all viruses of clinical or, more especially, veterinary significance. Examples of virus families that are of interest are listed below: Adenoviridae, Arenaviridae, Bunyaviridae, Calciviridae, Coronaviridae, Flaviviridae, Hepadnaviridae, Herpesviridae, Iridoviridae, Orthomyxoviridae, Paramyxoviridae, Papovaviridae, Parvoviridae, Pestiviridae, Picornaviridae, Poxviridae, Reoviridae, Retroviridae, Rhabdoviridae, and Togaviridae.

We envisage that, for viruses of veterinary significance, the sequestrating receptors of the invention may be used to generate transgenic livestock (farm animals, including fish, poultry and mammals or domestic pets) with engineered resistance to infection by the offending virus(es). Specific examples of viruses for which this would be useful are Swine Fever (Hog Cholera), Rabies, Enzootic Bovine Leucosis, Maedi/Visna, Sheep Pox, Foot and Mouth Disease, Teschen, Swine Vesicular Disease, Newcastle Disease, Rinderpest (Cattle Plague), African Swine Fever, Aujeszky's Disease (Pseudorabies).

A protective polypeptide will be considered to have “high binding affinity” for the component of the micro-organism if it is of the same order of magnitude as, or greater than, the binding affinity of the natural receptor for the micro-organism.

In preferred embodiments the protective polypeptide is a chimeric molecule, which typically will comprise at least a portion of the natural receptor for the micro-organism. Advantageously such a chimeric molecule will comprise at least an effective portion of the binding domain of the natural receptor for the micro-organism (an “effective portion” of the binding domain is a part sufficient to retain substantially all of the binding activity of the natural receptor).

Conveniently the protective polypeptide will prevent the micro-organism fusing with the host cell membrane. It is also generally preferred that the protective polypeptide will comprise a lysosomal targeting signal, such that microorganisms bound to the protective polypeptide may be directed to acidified lysosomes, wherein they will be degraded.

In a further aspect the invention provides a nucleic acid construct suitable for performing the method defined above, and for use of a nucleic acid construct in the method defined above. In another aspect the invention provides a cell into which has been introduced a nucleic acid in accordance with the method defined above. In yet another aspect the invention provides a method of making a composition for use in protecting eukaryotic cells against intracellular infection. The invention also provides a transgenic plant or animal having increased resistance to disease caused by a pathogenic micro-organism capable of existing intracellularly in the non-transgenic plant or animal, and to a method of making the same.

Suitable nucleic acid constructs, and methods of making them, will be apparent to the skilled in the art from the disclosure contained herein and with the benefit of the publications referred to herein. The constructs will preferably comprise a promoter and one or more regulatory signals operably linked to the coding sequence and active in the eukaryotic cell in question although such signals could, in theory, be provided by fortuitous insertion of the coding sequence at an appropriate site in the host cell genome.

Methods of introducing the nucleic acid construct into the host cell will be well-known to those skilled in the art and conveniently such introduction will be effected by way of transformation, although transduction, electroporation or “biolistic” methods and the like could be employed (especially with respect to introduction into plants cells).

For effective sequestration of the micro-organism away from its normal entry route into the cell, the protective receptors should preferably bind the micro-organism more avidly than the natural receptors. The affinity of the micro-organisms for the protective receptors should therefore be comparable to, or preferably higher than, their affinity for natural receptors. This might best be achieved by transplanting the natural micro-organism binding site into a chimaeric protective receptor, a strategy that would also prevent the emergence of a mutated micro-organism strain capable of binding selectively to the unmodified natural receptor. For high-avidity binding, the protective receptors should preferably also be expressed at higher density than the natural receptors and should advantageously be multimeric. They should also be more accessible for micro-organism attachment than the natural receptors, possibly by virtue of their ability to project a greater distance from the surface membrane of the cell.

Individual micro-organisms are likely to differ in respect of the type of protective receptor that will interfere with their efficient entry into target cells. Potentially important parameters relating to the protective receptors include: the distance that they project from the cell membrane: their flexibility or stiffness; their lateral associations with other constituents of the target cell membrane; their lateral mobility within the target cell membrane: their density of expression at the cell surface: and their tendency/ability to oligomerise or cluster in response to micro-organism attachment. In general, a set of parameters that differs greatly from those of the natural receptor, (in favour of binding to the micro-organism preferentially over the natural receptor) is likely to be more effective. For optimal efficiency of cell protection, the cytoplasmic domain of the protective receptor should carry a lysosomal targeting signal that promotes the rapid transport of sequestered micro-organisms to the lysosomal compartment for degradation.

Thus, for example, a protective receptor which, in relation to the natural receptor, is comparatively rigid and/or in which the binding site for the micro-organism projects further from the host cell, will tend to inhibit fusion of a virus with the host cell membrane.

The binding site may be extended further from the host cell membrane by inserting a spacer polypeptide between the binding site and the transmembrane domain which anchors the receptor in the host cell membrane. Conveniently the spacer polypeptide will comprise one or more domains, typically corresponding to extracellular domains from another membrane protein (preferably one normally expressed by the organism in which the host cell is usually found, thereby minimising the possibility of stimulating an imune rsponse to the chimeric protective receptor). An example of suitable domains for insertion are the immunoglobulin-like domains found in a number of membrane proteins (e.g. CD4). The number of domains required to be inserted optimally to prevent fusion will depend on the precise nature of the receptor molecule and the micro-organism in question, but such may be determined by those skilled in the art without requiring inventive effort, by simple exercise of trial and error.

In one particular embodiment, the invention provides a novel strategy for protecting permissive cells against infection by a retrovirus, such as HIV, by transducing them with a therapeutic gene encoding a chimaeric receptor molecule which incorporates a high-affinity binding site for the retroviral SU glycoprotein, but does not support virus entry. Retroviral particles approaching the surface of the genetically modified cells will be bound avidly by the chimaeric receptors and thereby sequestered away from wild type retroviral receptors, such that they cannot go on to infect the cells.

The above features will also be generally desirable in receptors designed to protect cells against intracellular infection by microorganisms other than retroviruses. Additional preferred features of the protective receptors of the present invention are that they should be non-immunogenic and should not interfere with the normal functions of the cells in which they have been expressed.

The chimaeric receptor genes of the present invention might be useful for antiviral gene therapy applications, for example to protect HIV-negative individuals against infection by HIV or to provide HIV-positive individuals with a reservoir of HIV-resistant T-lymphocytes and monocyte/macrophages, thereby limiting the progression of their HIV-induced immune deficiency. Autologous haemopoietic stem cells and/or their progeny could be transduced directly with the chimaeric receptor genes or transduced ex vivo, and reinfused.

The chimaeric receptor genes of the present invention might also be useful for generating HIV scavenging cells that avidly bind and sequester HIV particles. Such scavenging cells might be prepared ex vivo (autologous or allogeneic, live or irradiated) and administered to HIV-positive individuals to reduce their viral burden. Alternatively, cells that are normally nonpermissive for HIV (e.g. hepatocytes) might be converted to efficient HIV scavenging cells by direct in vivo gene delivery.

The feasibility of the approach defined above has now been demonstrated. Buchholz et al., (J. Virol. 1996 70, 3716-3723) showed that target cells expressing chimeric CD4/CD46 receptors for measles virus could be protected against infection: chimeric receptors, which presented the binding site for measles virus haemagglutinin protein at a site more distal to the host cell membrane than the natural CD46 receptor, allowed measles virus to bind (and indeed, even enhanced such binding) but prevented viral fusion with the host cell membrane.

### EXAMPLES

Design of genes coding for chimaeric receptors that will sequester HIV virions, thereby protecting HIV-susceptible target cells from infection by HIV.

### Example 1

### “Long” HIV Receptor

The normal function of CD4 is binding to MHC Class II which stabilises the interaction between T cell receptors and cognate class II MHC-peptide complexes. The length of the CD4 molecule is thought to be an important determinant of this normal function. Increasing the length of the CD4 receptor should therefore abrogate its normal function without diminishing its ability to bind to HIV gp120. It is anticipated that incoming HIV virions will attach preferentially to the long HIV receptors when they are co-expressed with unmodified CD4. However, the long HIV receptors would not be expected to interfere with the normal functions of the unmodified CD4 when co-expressed in the same cell.

The gene coding for CD4 is modified by the insertion, between the transmembrane domain and the adjacent extracellular domain (domain 4), of a sequence coding for four nonimmunogenic spacer domains. The spacer domains are functionally inert. Suitable spacer domains include the membrane proximal Ig-like domains from membrane receptors such as CD4, ICAM-1 and c-Kit, or the membrane proximal short consensus repeat (“SCR”) domains from membrane receptors such as CD46. Short linkers of three to five glycine residues may be inserted at newly created domain boundaries to allow for structural flexibility. The spacer domains should be derived from non-xenogenic proteins.

The newly constructed gene coding for a chimaeric receptor is cloned into an expression vector under the control of a strong promoter. The expression vector is introduced into a CD4-expressing mammalian cell line that is naturally susceptible to HIV infection and clones which express the chimaeric receptor are isolated. These clones, expressing a combination of CD4 and chimaeric CD4, are then challenged, either with HIV or with HIV-based retroviral vector particles carrying a marker gene. Susceptibility of the cells to infection by HIV is monitored by the transfer of the marker gene, or by following the spread of the HIV infection through the culture and the release of progeny virions from the infected culture using standard methodology. It is thereby demonstrated that, compared to the CD4-positive parental cells, the derivative clones expressing the chimaeric CD4 receptor are resistant to infection by HIV. HIV gp120 binding to the chimaeric receptors is monitored independently by FACS analysis using cells that express the chimaeric receptor in the absence of wild type CD4. It is thereby demonstrated that the chimaeric receptors can efficiently capture HIV virions and, in conjunction with the results of the previous experiments, this constitutes proof that the chimaeric receptor is protecting cells from infection by competitively sequestering the HIV virions that approach the target cell surface.

To demonstrate that the chimaeric CD4 receptor does not interfere with normal CD4 functions, it is expressed in antigen-specific helper T cell clones which are then tested for preservation of their normal reactivity to antigen presented in the context of autologous MHC ClassII-peptide complexes. For transfer of the chimaeric CD4 receptor gene to the helper T cell clones, retroviral vectors that transfer this gene along with a selectable marker gene are prepared using established methods and are used to infect the helper T cell clones, whereupon subclones expressing the chimaeric receptor are selected. The antigen reactivity of the selected subclone(s) is then tested using established methods.

### Example 2

### Long, Oligomeric HIV Receptor

The gene coding for this receptor is constructed as in the example above, except that it includes an additional domain that is capable of (homo)dimerisation. The dimerisation signal may be derived from a receptor such as the insulin receptor to induce the formation of stable disulphide-linked receptor dimers. Alternatively, the dimerisation signal might be a domain such as the fourth immunoglobulin domain of the stem cell factor receptor c-kit, which facilitates ligand-induced receptor dimerisation—the chimaeric receptor in this could undergo gp120-induced dimerisation to enhance the avidity of virus attachment. Other possibilities are to insert domains coding for short amphipathic helical peptides that are known to form stable dimeric leucine zipper structures.

Having made these constructs, they are tested as indicated in example 1.

### Example 3

### Long HIV Receptor with a Lysosomal Targeting Signal

In this case, the chimaeric receptor is constructed as in example 1, except that the cytoplasmic domain of CD4 is replaced by the cytoplasmic domain of the EGF receptor or other receptor protein tyrosine kinase since these cytoplasmic domains are known to contain a lysosomal targeting signal.

Having made these constructs, they are tested as indicated in example 1.

### Example 4

### Long, Oligomeric HIV Receptor with a Lysosomal Targeting Signal

In this case the chimaeric receptor is constructed incorporating the features described in examples 1, 2 and 3 above. It is then tested as indicated in example 1.

1. A method of inhibiting intracellular infection of a eukaryotic cell by a micro-organism comprising introducing into the cell a nucleic acid sequence directing the expression on the surface of the cell of a protective polypeptide receptor molecule which has high binding affinity for a component of a micro-organism capable of existing intracellularly, such that binding of the micro-organism to the protective polypeptide prevents productive infection of the cell by the micro-organism.

2. A method according to claim 1, wherein the micro-organism is a virus.

3. A method according to claim 1 or 2, wherein the micro-organism is a retrovirus.

4. A method according to any one of claims 1, 2 or 3, wherein the micro-organism is HIV type I or type II.

5. A method according to any one of the preceding claims, wherein binding of the micro-organism to the protective polypeptide receptor inhibits fusion of the micro-organism with the host cell.

6. A method according to any one of the preceding claims, wherein binding of the micro-organism to the protective polypeptide receptor directs the micro-organism to the endosomal compartment of the host cell.

7. A method according to any one of the preceding claims, wherein the protective polypeptide is a chimaeric molecule.

8. A method according to any one of the preceding claims, wherein the protective polypeptide comprises at least a portion of the natural receptor for the micro-organism.

9. A method according to any one of the preceding claims, wherein the protective polypeptide comprises an effective portion of the binding domain of the natural receptor for the micro-organism.

10. A method according to any one of the preceding claims, wherein the protective polypeptide comprises the V1 domain of CD4.

11. A method according to any one of the preceding claims, wherein the protective polypeptide comprises the V1 and V2 domains of CD4.

12. A method according to any one of the preceding claims, wherein the protective polypeptide comprises at least part of the EGF receptor.

13. A method according to any one of the preceding claims, wherein the protective polypeptide comprises a lysosomal targeting signal directing bound micro-organisms to a lysosomal compartment for degradation.

14. A method according to any one of the preceding claims, wherein the protective polypeptide is expressed at sufficient level such that there is a greater number of protective binding sites on the surface of the cell than natural binding sites for the micro-organism.

15. A method according to any one of the preceding claims, wherein the protective polypeptide is multimeric.

16. A method according to any one of the preceding claims, wherein the micro-organism attachment site on the protective polypeptide projects further from the cell surface than the attachment site on the natural receptor for the micro-organism.

17. A method according to any one of the preceding claims, wherein the protective polypeptide is a chimeric polypeptide and comprises an extracellular spacer polypeptide inserted between a micro-organism attachment site and a transmembrane portion.

18. A method according to any one of the preceding claims, wherein the nucleic acid is introduced into cells which are inherently incapable of supporting productive infection by the micro-organism.

19. A method according to any one of the preceding claims, wherein the nucleic acid is introduced into the cell ex vivo.

20. A nucleic acid construct for use in the method of any one of claims 1 to 19.

21. Use of a nucleic acid construct for the method of any one of claims 1 to 19.

22. An eukaryotic cell or the progeny thereof, into which has been introduced a nucleic acid sequence in accordance with the method of any one of claims 1 to 19.

23. A transgenic plant or animal having increased resistance to a disease caused by a pathogenic micro-organism normally capable of existing intracellularly in the non-transgenic plant or animal, the transgenic plant or animal being grown from a eukaryotic cell into which has been introduced a nucleic acid sequence directing the expression on the surface of the cell of a protective polypeptide receptor molecule which has high binding affinity for a component of the pathogenic micro-organism, such that binding of the microorganism to the protective polypeptide prevents productive infection of the cell by the micro-organism.

24. A method of making a composition for use in protecting an eukaryotic cell against intracellular infection, comprising mixing a nucleic acid sequence directing the expression on the surface of the cell of a protective polypeptide receptor molecule, with a physiologically acceptable carrier substance.

25. A method of making a transgenic plant or animal having increased resistance to a disease caused by a pathogenic micro-organism normally capable of existing intracellularly in the non-transgenic plant or animal, the method comprising: introducing into a plant or animal cell a nucleic acid sequence directing the expression on the surface of the cell of a protective polypeptide receptor molecule which has high binding affinity for a component of the pathogenic micro-organism, such that binding of the micro-organism to the protective polypeptide prevents productive infection of the cell by the micro-organism; and growing the plant or animal cell into a transgenic plant or animal.
